(19) United States
(12) Patent Application Publication
(10) Pub. No.: US 2010/0170506 A1
(43) Pub. Date: Jul. 8, 2010

(54) Oral device to facilitate the artificial pulmonary ventilation to an unconscious edentulous patients

(76) Inventors: Andres Pawels, Bogata (CO); Claudia Nino De Mejia, Bogata (CO); Enrique Arango Galvis, Bogata (CO); Fernando Raffan Sanabria, Bogata (CO)

Correspondence Address: ABELMAN, FRAYNE & SCHWAB, 666 THIRD AVENUE, 10TH FLOOR, NEW YORK, NY, 10017, US

(21) Appl. No.: 12/589,875

(22) Filed: Oct. 28, 2009

(30) Foreign Application Priority Data
Dec. 5, 2008 (CO) 08-129703

(51) Int. Cl. A61M 16/04

(52) U.S. Cl. 128/200.26; 128/207.14

(57) ABSTRACT

A helper device for placement into a mouth cavity to facilitate the artificial pulmonary ventilation for an unconscious edentulous patient comprises a U shaped plate, which comprises upper, lower and side coupling surfaces for intra-mouth fixation, as well as a configured central orifice for housing an oropharynx cannula. Such device allows medical personnel to control and perform ventilation operations while the patient is wearing a facemask.

### BACKGROUND OF THE INVENTION

This invention refers to artificial ventilation devices to facilitate face mask ventilation in unconscious edentulous patients, and more specifically it refers to those devices designed to be placed into the oropharynx region of the patient, with the purpose of preventing an air-inlet obstruction and also to permit spontaneous and controlled ventilation.

When doctors related to the handling of airways management are inquired about the principal causes resulting in problems and complications of patients, they disclose the difficulty or inability to keep an oxygen saturation (O₂) of more than 92% using 100% oxygen, due to: the loss or leak of gas through the face mask, the permanent need of increasing the fresh gas (O₂) up to 15 l/min, use the high pressure valve several times while the patient is assisted, the difficult to achieve thorax motions, the requirement of more than two people to keep the mask properly, change the operator due to fatigue for holding the mask.

Due to above reasons, there exists currently a growing need and interest for developing devices facilitating the endo-tracheal intubations operations, reducing at the same time the complications arising in distressing situations. However, the incidence of hard ventilation patients (5%) is much higher than the difficult intubations incidence (2.8%), and the intubations device is aimed to this section of the algorithm of difficult airway.

The maintenance of airway permeability and oxygenation are the principal objectives of ventilation with facemask. To facilitate the difficult airway management, and reduce the incidence of severe adverse events, some practical guidelines and algorithms have been established, while intensive works are done in the design of strategies and devices, which may facilitate the airway management operations.

Constructing the first step in the airway control, before securing a definite airway, with endo-tracheal intubations, which consists of the placement of a flexible gum or plastic tube inside the trachea, generally using an inflating balloon around the distal ends. In emergency cases, in unconscious patients, or during the induction of the general anesthesia, time is a matter of essence to assure an adequate oxygenation and ventilation. The definite placement of a tube inside the trachea requires special abilities, training, and it can result in damages to the soft tissues or to the larynx structure, thus increasing the degree of difficulty for the managing of ventilation and placing the patient's life in risk.

Alternatively, it is possible to use an oro-pharynx or naso-pharynx air inlet consisting of a tube from the mouth or nose to the pharynx, but not inside the larynx, which is used close to a facemask, unlike the endotracheal tube. In spite that the obstruction is prevented, the oro-pharynx or naso-pharynx air intake cannot be properly used with any type of patient. For instance, it is not adequate to use with patients with difficult ventilation predictors, with urgent needs of brain-cardio-pulmonary reanimation, such as bearded patients on which a conventional face mask would not produce any seal with the face edge and thus it is not possible to provide the proper air intake, or with edentulous patients where an oropharynx cannula is not enough as it makes no seal to the mouth to assure the intake of the adequate quantity of air for the patient's reanimation.

The previous art, for example, defines some devices such as the British Patent No. 2111394B, which defines an artificial ventilation device, which includes a curved or flexible tube, and a portion of mask in one of the tube ends. The mask portion features a flexible ring peripheral formation which can be inflated, surrounded an inner hollowed space or lumen of the mask portion. Said peripheral ring formation of the mask portion is previously made in a stiff elliptical shape, able to be easily attached or loosened in the existing space behind the larynx, so that it couples around the circumference of the larynx inlet, without permitting the device to penetrate into the larynx, where the tube is communicated with the lumen of the mask portion to permit the ventilation with the tube axis principally aligned to the length of the peripheral elliptical ring and stiff formation of the mask portion. The device thus makes a larynx mask. This device has been successfully tested in practice, but it will not afford its use in edentulous patients, so that such devices are not adequate to use in emergency reanimation methods for assuring the sealing at the mouth inlet.

It is therefore necessary to have a ventilation device for the management of the airway, facilitating the control and performance of ventilation operations with a face mask in patients who particularly have ventilation problems, such as those edentulous patients, bearded patients, obese patients or those having maxillary anatomical alterations.

Particularly, the previous art does not illustrate any device that when placed inside the patient mouth cavity may allow the ventilation using a face mask during the general anesthesia induction, and in procedures of cerebrum-cardio-pulmonary reanimation, especially with patients with difficult ventilation predictors and generally in all those individuals featuring anatomical alterations where it is difficult to attain a proper sealing between the face mask and the patient's face.

### SUMMARY OF THE INVENTION

Therefore, one first objective of this invention is avoiding the disadvantages of the previous art. Particularly, the object of this invention is creating a device of inclusion inside the oral cavity to facilitate the artificial pulmonary ventilation for an unconscious edentulous patient, with whom ventilation operations are controlled and carried out using a facemask.

A no less important objective is that the invention configuration, due to its simplicity, is easy to manipulate in medical emergencies such as emergency on the site of the event and the like.

Additionally, there is the need of creating a universal device, coupled to any means of ventilation supply, regardless of conditions, such as bear of the patients or edentulous conditions.

Therefore, this invention is intended to temporarily restore the normal anatomy, in case of edentulous patients, or correct it in other types of patients, with the purpose of permitting an adequate sealing of the face mask floater against the face, thus permitting to generate a positive pressure in the patient airway, preventing the leaking of medicinal or anesthetic gases, assuring thus the permeability of the airway, the adequate oxygenation and reducing the difficulties during the ventilation.

This invention complies with the above needs and provides other related advantages.

The novelty characteristics that are considered as a fundamental of the invention are particularly explained in the appended claims. The additional advantages thereof will be better understood on the detailed description with the preferred modalities and the proper reference to the accompanying drawings.

### BRIEF DESCRIPTION OF THE DRAWINGS

To further clarify the invention and the advantages thereof compared to the known art, there appears below a description with the help of the attached drawings, the possible forms of illustrations but not limitations to the application of such principles.

FIG. 1. Thus figure shows a front view of the device to facilitate the pulmonary artificial ventilation of the invention herein.

FIG. 2. This figure illustrates in detail a top view of the device to facilitate the pulmonary artificial ventilation of the invention herein.

FIG. 3. This figure illustrates an isometric view of the device to facilitate the pulmonary artificial ventilation of the invention herein, which includes an oropharynx cannula in its arrangement for use.

### DETAILED DESCRIPTION OF THE INVENTION

This invention is related to a helper device of inclusion inside the oral cavity to facilitate the pulmonary artificial ventilation for an unconscious edentulous patient, with which ventilation operations are controlled and performed with a facemask.

This invention has an application in different areas of the patients' care. In areas of pre-hospital management and patient's transport, emergencies, intensive care units, patients under intra hospital brain-cardio-pulmonary reanimation, and also in the area of operating rooms in patients undergoing a general anesthesia.

The fault in the management of the airway is one of the most relevant factors related to the morbidity and mortality of patients under a general anesthesia condition.

To facilitate the management of the airway and to reduce the incidence of adverse effects during the management period, the device of this invention has been defined.

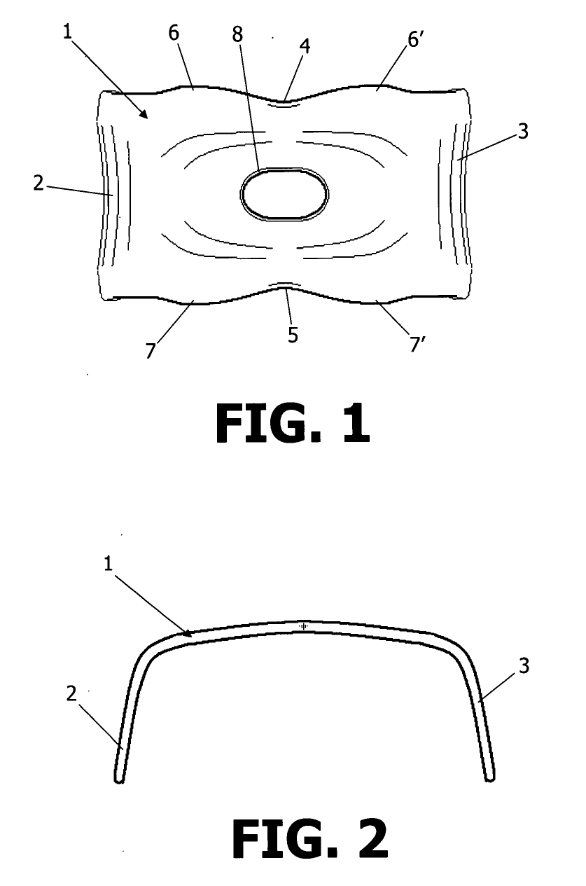

Making a detailed reference to the drawings, particularly in FIGS. 1 and 2, the helper device comprises a plate 1 of a U extended configuration.

Said plate 1 comprises side left and right regions making a left extension fin 2 and a right extension fin 3 for its placement inside the maxillary vestibule region.

Said plate 1 includes additionally upper and lower coupling surfaces comprising an upper inlet 4 and a lower inlet 5 to assist in the mouth inner subjection between the region of gums and the lips, where each inlet of the device is located in each lip frenum assuring its side immobility within the mouth cavity and protecting at the same time easy to injure anatomical structures such as the lip frenums.

Likewise, the device of the invention comprises side subjection means comprising an upper left side protrusion 6, a lower left side protrusion 7; an upper right side protrusion 6′; and a lower right side protrusion 7′.

Starting from said sided subjection means, each protrusion is interposed between the gums in the mouth side region, thus adjusting the device.

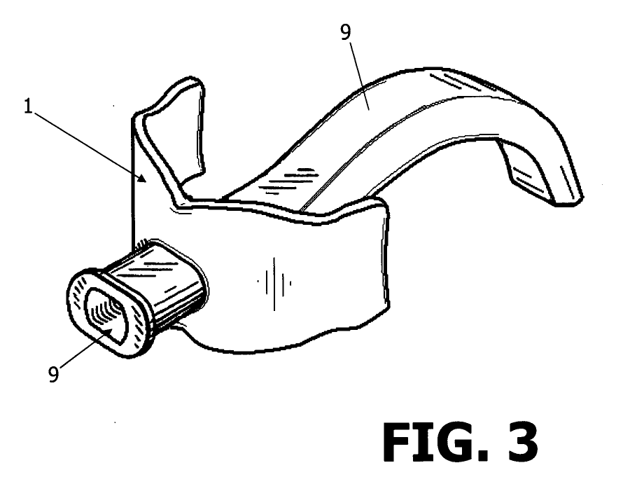

Also, the device of this invention comprises a central orifice 8 configured for introducing a conventional oro pharynx cannula 9 as illustrated in FIG. 3.

Said orifice 8, permits the ingress of said universal cannula, so that with the physician's ability, he/she may choose the cannula adequate to the patient in terms of measurement and size, to be inserted into the device of the invention, adjusting it within the mouth cavity to proceed with the adequate ventilation in relation to the reanimation process.

According to preferred modalities, the material for the preparation of the device of the invention herein may comprise plastics, PVC or similar, with the adequate characteristics of medical devices.

This invention is generally applicable to the field of Medicine for its special use in the field of anesthesiology, critical care, emergencies and all those fields where it is necessary to care for patients, requiring manual ventilation with positive pressure, with risk factors for difficult ventilation, with the purpose of preventing gas leaks (oxygen-air) during the process of manual ventilation assistance.

When a patient suffers alterations of his/her consciousness status, either induced by pharmaceuticals or not, the loss of protective reflexes requires the assistance to maintain the patient's airway permeable by the application of positive pressure over the mouth or nose with the purpose of ventilating them, so as to being enabled to administer oxygen in sufficient quantities reaching the pulmonary alveoli and the blood circulation.

This invention complies with its principal objective in general, with the interaction of a duly trained operator (medical staff or paramedical staff); a device delivering oxygen with positive pressure (AMBU-Jackson-Reses-etc); one face mask to cover the nose and mouth; and a device to make the airway permeable (i.e. oro-pharynx cannula or naso-pharynx cannula).

Therefore, this invention is applicable to a great number of patients (3-5%) presenting anatomical conditions such as face hair, beard, edentulous, making its ventilation difficult with positive pressure, placing them at risk of suffering a permanent neurological damage or even death by hypoxemia while a definite airway is achieved.

Only some preferred modalities of the invention have been illustrated by way of example. To this regard, it shall be appreciated that the construction of the helper device placed into the mouth cavity to ease the artificial pulmonary ventilation, as well as the arrangements in its specific configuration, can be chosen from a set of options without being separated from the spirit of the invention, as per the following claims.

1. An inclusion helper device for insertion into a human mouth cavity to ease the artificial pulmonary ventilation in edentulous patients, comprising a plate 1 having an extended U configuration, with left side and right side regions in the form of left extension fins (2) and right side fins (3) to be located inside the mouth; wherein said device further includes upper and lower coupling surfaces comprising an upper inlet (4) and a lower inlet (5); and wherein said device additionally comprises side subjections comprising an upper left side protrusion (6), a lower left side protrusion (7), an upper right side protrusion (6′); and a lower right side protrusion (7′).

2. An inclusion helper device according to claim 1 wherein said upper inlet (4) and said lower inlet (5) are configured to help to the subjection to the inner part of the patient's mouth.

3. An inclusion helper device according to claim 1, wherein each protrusion is configured to be interposed between the gums in the side region of the patient's mouth for the device adjustment.

4. An inclusion helper device according to claim 1, further comprising a central orifice (8) configured to receive a conventional oro pharynx cannula (9).

5. An inclusion helper device according to claim 4, wherein said orifice (8) permits the inlet of said universal cannula, chosen from the measurement and size appropriate to the patient, to be inserted in the device, for adjusting it to the mouth cavity.

6. An inclusion helper device according to claim 1, wherein said device is formed of plastics, PVC or a similar material with characteristics appropriate for medical devices to be inserted in a human mouth.
